(19) United States
(12) Patent Application Publication
(10) Pub. No.: US 2014/0065227 A1
(43) Pub. Date: Mar. 6, 2014

(54) NEURAL STEM CELLS DERIVED FROM INDUCED PLURIPOTENT STEM CELLS

(71) Applicants: WHITEHEAD INSTITUTE FOR BIOMEDICAL RESEARCH, Cambridge, MA (US); THE MCLEAN HOSPITAL CORPORATION, Belmont, MA (US)

(72) Inventors: Ole Isacson, Cambridge, MA (US); Jan Pruzak, Cambridge, MA (US); Marius Wernig, Woodside, CA (US); Rudolf Jaenisch, Brookline, MA (US)

(21) Appl. No.: 13/920,314

(22) Filed: Jun. 18, 2013

Related U.S. Application Data
(63) Continuation of application No. 12/419,222, filed on Apr. 6, 2009, now abandoned.
(60) Provisional application No. 61/043,085, filed on Apr. 7, 2008.

(51) Int. Cl. A61K 35/30

(52) U.S. Cl. CPC A61K 35/30
USPC 424/490; 435/377; 424/93.7

(57) ABSTRACT

The present invention provides novel populations of neural stem cells derived from induced pluripotent stem cells, and methods for making and using the same.

### CROSS-REFERENCE TO RELATED APPLICATIONS

This application is a Continuation application of co-pending U.S. patent application Ser. No. 12/419,222 filed on Apr. 6, 2009, which claims benefit under 35 U.S.C. §119(e) of U.S. Provisional Patent Application No. 61/043,085, filed on Apr. 7, 2008, the contents of each of which are hereby incorporated by reference in their entireties.

### GOVERNMENT SUPPORT

This invention was made with Government support under Grant No. NS039793 and 81XWH-05-1-0555 awarded by the National Institutes of Health and the U.S. Department of the Army. The Government has certain rights in this invention.

### FIELD OF INVENTION

This invention relates to the field of stem cells. Specifically, the invention provides methods for generating pluripotent cells from fibroblasts and inducing those cells to differentiate into neuronal phenotypes.

### BACKGROUND OF INVENTION

The following discussion of the background of the invention is merely provided to aid the reader in understanding the invention and is not admitted to describe or constitute prior art to the present invention.

A variety of neurodegenerative diseases are characterized by neuronal cell loss. The regenerative capacity of the adult brain is very limited. Mature neurons are believed to be post mitotic and there does not appear to be significant intrinsic regenerative capacity in response to brain injury and neurodegenerative disease. Further, pharmacological interventions often become increasingly less effective as the susceptible neuronal populations are progressively lost.

Cell transplantation therapies have been used to treat neurodegenerative disease, including Parkinson's disease, with moderate success (e.g., Bjorklund et al., Nat. Neurosci. 3: 537-544, 2000). However, wide-spread application of cell-based therapies will depend upon the availability of sufficient amounts of neuronal precursor cells.

Embryonic stem (ES) cells can be expanded to virtually unlimited numbers and have the potential to generate all cell types in culture. Therefore, ES cells are an attractive new donor source for transplantation and hold promise to revolutionize regenerative medicine. The ES cell based therapy is complicated, however, by immune rejection due to immunological incompatibility between patient and donor ES cells. The successful generation of cloned stem cells and animals by somatic cell nuclear transfer (SCNT) created the possibility to generate genetically identical “customized” SCNT-ES cells by using donor cells from a patient as the source of the nucleus (Hochedlinger et al., N. Engl. J. Med. 349: 275-286, 2003). This strategy would eliminate the requirement for immune suppression. Despite successful application of SCNT-ES cells in animal disease models, both technical and logistic impediments as well as ethical considerations of the nuclear transfer procedure complicate the practical realization of ‘therapeutic SCNT’ in human.

The ultimate goal of somatic reprogramming is to generate in vitro functional cell types relevant for therapy (e.g., neurons, cardiomyocytes, insulin-producing cells, hematopoietic cells). Recently, in vitro reprogramming of mouse fibroblasts into pluripotent stem cells (“iPS” cells), was achieved through retroviral transduction of the four transcription factors Oct4, Sox2, c-Myc and Klf4 and selection for reactivation of the ES cell marker gene Fbx15 (Takahashi et al., Cell, 126: 663-676, 2006). When selected for endogenous re-expression of the key pluripotency genes Oct4 or Nanog, reprogrammed fibroblasts were indistinguishable from blastocyst-derived embryonic stem cells both in terms of their epigenetic state and their developmental potential (Maherali et al., Cell Stem Cell 1: 55-70, 2007; Okita et al., Nature, 448: 313-317, 2007; Wernig et al., Nature, 448: 318-324, 2007). Importantly, iPS cells with a similar developmental potential can be generated from fibroblasts after transduction of the four genes by subcloning of colonies based on morphological criteria alone which allows the direct reprogramming of genetically unmodified fibroblasts (Meissner et al., Nat. Biotechnol. 25: 1177-1181, 2007). The therapeutic benefit of iPS cell-derived hematopoietic cells was recently demonstrated in a humanized mouse model of sickle cell anemia (Hanna et al., Science, 318: 1920-1923, 2007).

### SUMMARY OF THE INVENTION

The present invention is based on the discovery, isolation, and characterization of specific neural stem cell populations that are derived in vitro from induced pluripotent (iPS) cells, and methods for making and using the same.

In one aspect, the invention provides a method for producing neural stem cells by providing a pluripotent stem cells derived from mesenchymal cells (e.g., by overexpressing in the mesenchymal cells at least one transcription factor selected from the group consisting of Oct4, Sox2, c-Myc and Klf4); and obtaining the neural stem cells by culturing the induced pluripotent stem cells in the presence of at least one neural selection factor. In one embodiment, the method overexpresses, in mesenchymal cells (e.g., fibroblasts), at least two, three, or four transcription factors selected from the group consisting of Oct4, Sox2, c-Myc and Klf4. Optionally, the population of iPS cells may be selected or refined (e.g., depleted or enriched) for certain cell types prior to culturing in the presence of growth factors. For example, the iPS cells may be selected for expression of Fbx15, Oct4, Klf4, and/or Nanog.

Neural selection factors include, for example, sonic hedgehog (SHH), fibroblast growth factor-2 (FGF-2), and fibroblast growth factor-8 (FGF-8), which may be used alone or in pairwise combination, or all three factors may be used together. In one specific embodiment, the iPS cells are cultured in the presence of at least SHH and FGF-8. In another embodiment, FGF-2 is omitted. Preferred mesenchymal cells are fibroblasts including, for example, skin fibroblasts, and liver cells (e.g., hepatocytes). Preferably, the mesenchymal cells are mammalian cells including, for example, human cells. Preferably, the neural stem cells derived from the iPS cells express nestin. In some embodiments, the pluripotent stem cells are cultured in the presence of the one or more neural selection factors for 4, 5, 6, 7, 8, 9, 10, 12, 14, 16, 18, 20 days or more.

In another aspect, the invention provides a population of neural stem cells produced by any of the foregoing methods. Preferably, the population of neural stem cells is characterized in that at least 50%, 75%, 85%, 90%, 95%, or 99% of the cells of the population expresses nestin. Preferably, the nestin-expressing cells further express at least one of En-1, Pitx3, and Nurr-1. In other preferred embodiments, the population of neural stem cells has been depleted of at least 50%, 75%, 85%, 95%, or 99% of the cells expressing surface markers of immature embryonic stem cells including, for example, SSEA-1, SSEA-3, SSEA-4, Tra-1-81, and Tra-1-60. Preferably, the population of neural stem cells contains less than 10%, less than 5%, less than 2.5%, less than 1%, or less than 0.1% of cells that express the selected marker (e.g., SSEA-4).

In another aspect, the invention provides a population of early neurons produced by any of the foregoing methods. In one embodiment, the iPS-derived neural stem cells are cultured in the presence of at least one of sonic hedgehog (SHH), fibroblast growth factor-8 (FGF-8), basic fibroblast growth factor (bFGF), and brain-derived neurotrophic factor (BDNF), in order to produce the early neurons. Preferably, the early neurons express at least one of tyrosine hydroxylase, DAT, and VMAT. Exemplary culture methods for producing early neurons from neural stem cells (including iPS-derived neural stem cells) are disclosed in Pruszak et al. (Stem Cells 25: 2257-2268, 2007) and Sonntag et al. (Stem Cells 25: 411-418, 2006). Preferably, the iPS-derived neural stem cells are cultured in the presence of two, three, or all four of the neural selection factors. Preferably, the population of early neurons is characterized in that at least 50%, 75%, 85%, 90%, 95%, or 99% of the cells of the population expresses tyrosine hydroxylase. In other preferred embodiments, the population of early neurons has been depleted of at least 50%, 75%, 85%, 95%, or 99% of the cells expressing surface markers of immature embryonic stem cells including, for example, SSEA-1, SSEA-3, SSEA-4, Tra-1-81, and Tra-1-60. Preferably, the population of early neurons contains less than 10%, less than 5%, less than 2.5%, less than 1%, or less than 0.1% of the cells that express the selected marker (e.g., SSEA-4).

In another aspect, the invention provides a therapeutic composition containing cells produced by any of the foregoing methods or containing any of the foregoing cell populations. Preferably, the therapeutic compositions further comprise a physiologically compatible solution including, for example, artificial cerebrospinal fluid or phosphate-buffered saline. In other embodiments, the cells contained in the therapeutic composition are encapsulated.

In another aspect, the invention provides a method for treating a neurodegenerative disease (e.g., Parkinson's disease) in a patient by administering to the brain of said patient any of the foregoing therapeutic compositions. The therapeutic compositions may be administered to the patient by any appropriate route. Preferably, the therapeutic compositions are injected into the caudate nucleus or the midbrain of the patient.

The term “induce pluripotent stem cell” (iPS cell) refers to pluripotent cells derived from mesenchymal cells (e.g., fibroblasts and liver cells) through the overexpression of one or more transcription factors. In one specific embodiment, iPS cells are derived from fibroblasts by the overexpression of Oct4, Sox2, c-Myc and Klf4 according to the methods described in Takahashi et al. (Cell, 126: 663-676, 2006), for example. Other methods for producing iPS cells are described, for example, in Takahashi et al. (Cell, 131: 861-872, 2007) and Nakagawa et al. (Nat. Biotechnol. 26: 101-106, 2008). The iPS cells of the invention are also capable of cell division.

As used herein, “cells derived from an iPS cell” refers to cells that are either pluripotent or terminally differentiated as a result of the in vitro culturing or in vivo transplantation of iPS cells. “Cells derived from an iPS cell” specifically include neural stem cells and early neurons produced according to the principles of this invention.

As used herein, “neural stem cells” refers to a subset of pluripotent cells which have partially differentiated along a neural cell pathway and express some neural markers including, for example, nestin. Neural stem cells may differentiate into neurons or glial cells (e.g., astrocytes and oligodendrocytes). Thus, “neural stem cells derived from iPS cells” refers to cells that are pluripotent but have partially differentiated along a neural cell pathway (i.e., express some neural cell markers), and themselves are the result of in vitro or in vivo differentiation iPS cells.

As used herein, “early neurons” refers to a subset of cells which are more differentiated than neural stem cells and express some late-stage neuronal markers characteristic of a mature neuronal phenotype. Late-stage neuronal markers include, for example, TH, DAT, and VMAT.

As used herein, “SSEA-1” refers to the cell surface antigen commonly known as CD15, the Lewis-X antigen, and/or 3-fucosyl-N-acetyl-lactosamine in mice. The human homolog of SSEA-1 is known as SSEA-4.

As used herein, a population of cells that has been “depleted of cells expressing surface markers of immature embryonic stem cells” refers to a cell population that has undergone a selection process that removes at least some of the most immature pluripotent cells. Such cells express, for example, SSEA-1, SSEA-3, SSEA-4, Tra-1-81, and/or Tra-1-60. This selection process may be done by any appropriate method that preserves the viability of the more mature pluripotent cells that do not express the selection marker including, for example, fluorescence-activated cells sorting (FACS) or magnetically-activated cells sorting (MACS). Preferably, depleted populations contain less than 10%, less than 5%, less than 2.5%, less than 1%, or less than 0.1% immature pluripotent cells expressing the selection marker.

### BRIEF DESCRIPTION OF THE FIGURES

FIGS. 1A-1L show the morphological and neurochemical features of differentiated iPS cells derived from the O9 cell line. FIG. 1A shows undifferentiated iPS cells growing on a MEF feeder layer. FIG. 1B shows neural precursor cells growing in FGF2 containing media. FIG. 1C shows differentiated neural morphologies of iPS cells seven days after withdrawal of FGF2. FIG. 1D shows that a fraction of iPS-derived cells having a neuronal morphology are double-labeled for β-III-tubulin and TH, 7 days after withdrawal of the growth factors FGF2, FGF8, Shh, and in the presence of ascorbic acid. FIG. 1E shows that at the same stage (7 day factor withdrawal) many non-neuronal cells express the astrocytic marker GFAP. FIG. 1F shows a rare 04-positive oligodendrocytes found in this growth condition. FIG. 1G shows that the fraction of TH-positive cells over β-III-tubulin-positive cells increases during neuronal differentiation (error bars show the standard deviation of cell counts of three independent experiments). FIG. 1H shows that the vast majority of TH-immunoreactive cells coexpress En1, Pitx3, and Nurr1. FIG. 1I shows the coexpression of En1 and TH in iPS-derived cells having a neuronal morphology. FIG. 1J shows that most TH-positive neurons are co-labeled with antibodies against VMAT2. FIGS. 1K-1L show that most TH-positive cells are also positive for Pitx3 and Nurr1 seven days after withdrawal of the growth factors. Scale bar represents 200 μm for (a) and (b), 100 μm for (c), (d), (i),and (j), 50 μm for (e) and (k), and 20 μm for (f) and (l).

FIGS. 2A-2G show the extensive migration and differentiation of iPS cell-derived neural precursor cells in the embryonic brain. FIG. 2A shows transplanted cells which form an intraventricular cluster (left) and migrate extensively into the tectum four weeks after transplantation into the lateral brain ventricles of E13.5 mouse embryos. FIG. 2B shows a high density of integrated astrocyte-like cells in the hypothalamus. FIG. 2C shows the complex neuronal morphologies of GFP-positive cells in the septum. FIG. 2D is a confocal reconstruction of grafted GFP-fluorescent cells in the tectum with neuronal and glial morphologies. FIG. 2E shows the GFP immunofluorescence and a confocal reconstruction of an astrocytic cell and a long neuronal process. FIG. 2F shows the GFP-immunoreactive of a fine neuronal (presumably dendritic) processes. FIG. 2G is a schematic representation of the main integration sites of iPS cell-derived neurons and glia. Brain areas showing the highest contribution are midbrain, hypthalamus and septum. See Table 1 for more details. Scale bar represents 200 μm for (a)-(c), 100 μm for (d) and (f), and 50 μm for (e).

FIGS. 3A-3F show microscopic examination of cells. FIG. 3A shows a confocal reconstruction of a GFP-positive cell in the midbrain expressing the nuclear neuronal marker protein NeuN, 4 weeks after intrauterine transplantation. FIG. 3B shows another transplanted neuron expressing cytoplasmatic β-III-tubulin. FIG. 3C shows other cells colabeled with GFAP antibodies after projection of a stack of confocal sections. FIG. 3D shows that both host neurons and transplanted cells express the glutamate transporter EAAC1. FIG. 3E shows that the soma of grafted cells are labeled with antibodies against GAD67. FIG. 3F shows that TH-immunoreactivity is present in both host and grafted neurons. Scale bar represents 100 μm for (a)-(c) and 50 μm for (d)-(f).

FIGS. 4A-4G show morphological and functional analysis of cells. FIG. 4A is a high resolution photomicrograph of GFP-immunofluorescence showing the dendritic morphologies of transplanted neurons. FIG. 4B is a higher magnification of the region indicated in FIG. 4A, showing the presence of synaptic spines along this dendrite. FIG. 4C shows that integrated GFP-positive neurons are adjacent to many synaptophysin-positive patches indicating the presence of synaptic contacts from host axon terminals. FIG. 4D shows a GFP-expressing neuron (arrow) in acute slices of the dorsal midbrain of a P20 mouse after in utero transplantation. FIG. 4E shows GFP-positive neurons by infrared differential interference contrast (IR DIC) (arrow) and approached by a recording electrode (left). The trace (below) indicates spontaneous generation of action potentials. FIG. 4F shows the results of a voltage-clamp recording at −70 mV in extracellular solution containing 3mM Mg²⁺. Traces show spontaneous slow and fast currents that indicate that this transplanted neuron receives synaptic contacts from host cells. All 6 recorded GFP-positive neurons from two mice (age P20 and P22) exhibited similar spontaneous currents. FIG. 4G shows current-clamp recordings during current injection. Top traces represent superimposed membrane potential changes which demonstrates the capability of the grafted neurons to fire action potentials in response to a series of current injection (bottom traces) from a holding potential of −68mV. All 6 analyzed GFP-neurons showed these active membrane characteristic. Scale bars: 20 μm.

FIGS. 5A-5G show microscopic and functional studies of cells. FIG. 5A is a low power photomicrograph of an iPS cell graft, stained for TH, four weeks after transplantation into the rat brain receiving a unilateral 6-OHDA lesion. FIG. 5B is a higher magnification photomicrograph of another graft showing TH-positive soma and the dense innervation of the surrounding host striatum by donor-derived neurites (arrowheads). The dashed line indicates the edges of the graft. FIG. 5C shows that amphetamine-induced rotations (total rotations in 90 min after amphetamine injection) are significantly reduced in animals grafted with unsorted iPS cell populations (n=5) compared to the sham control animals (n=10) (p=0.0185). FIG. 5D shows that amphetamine-induced rotations in animals transplanted with iPS cell cultures after elimination of SSEA1-positive cells by FACS (n=4) are significantly reduced compared to control animals (n=10) (p=0.006). FIGS. 5E-5G are photomicrographs showing that the grafted TH-positive cells are co-labeled with antibodies against other dopaminergic markers including VMAT2, DAT, and En1. Scale bars: 50 μm.

FIGS. 6A-6H show morphological analysis of iPS cell-derived neural precursor cells. FIG. 6A shows that iPS cell-derived neural precursor cells grown in FGF2-containing media morphological characteristics of neural precursor cells. FIG. 6B shows that the cells adopt a more differentiated morphology six days after withdrawal of FGF2. The FGF2-responsive cells express the neural precursor cell markers Nestin (FIGS. 6C-6D), Sox2 (FIGS. 6E-6F), and Brn2 (FIGS. 6G-6H). FIGS. 6C, 6E, and 6G represent Dapi-stained micrographs of the corresponding visual field. Scale bar: 100 μm.

FIGS. 7A-7J are photomicrographs of an iPS cell graft. FIG. 7A is a low power photomicrograph of an H&E-stained iPS cell graft which partly consists of a tumor showing signs of non-neural differentiation indicating the formation of a mature teratoma. FIG. 7B is a higher magnification of the same tumor showing squameous epithelium and salivary gland structures (inset). FIG. 7C-7D shows groups of cells in the teratoma that are immunoreactive with antibodies against SSEA1 adjacent to neurons expressing TH. Cell nuclei are stained with DAPI. FIG. 7E shows that the tumors contain epithelial cells which express cytokeratin and doublecortin (DC). FIG. 7F shows other cellular structures are Villin-positive. FIG. 7G shows the presence of undifferentiated iPS cell colonies in neuronal cultures over 3 weeks after the induction of differentiation. FIG. 7H shows the corresponding DAPI staining. FIG. 7I shows that undifferentiated colonies are immunoreactive with Nanog antibodies. FIG. 7J shows the relative expression levels of viral transcripts using quantitative PCR analysis in uninfected MEFs, MEFs two days after infection with the 4 viruses, the Oct4-neo selected iPS cell line O9, and in a teratoma (Neu-T) which had formed 4 weeks after transplantation of unsorted, differentiated iPS cells enriched for dopamine neurons. Scale bars represent 500 μm for (b), 50 μm for (c)-(f), and 100 μm for (g)-(i).

FIGS. 8A-8C show the FACS sorting results. FIG. 8A shows the FACS sorting results of SSEA1 expression in neuronal cultures 5 days after withdrawal of Shh, FGF8 and FGF2 before sort (left panel) and in the 2 sorted populations (right panels). FIG. 8B shows that SSEA1-negative sorted cells (right) displayed mostly neural morphologies when plated onto tissue culture dishes, whereas the SSEA1-positive sorted cells (left) exhibited an undifferentiated ES cell morphology. FIG. 8C is a photomicrograph showing that a graft of SSEA1-negative sorted cells was smaller than that of unsorted cells and contained TH-positive neurons extending long neurites into the host striatum (arrowheads). No teratoma formation was observed in any of the 4 transplanted animals up to 8 weeks after transplantation. Scale bar: 100 μm.

### DETAILED DESCRIPTION OF INVENTION

The present invention provides novel populations of neural cells differentiated from mesenchymal cell-derived pluripotent stem cells, and methods for making and using the same. The inventive cells are either pluripotent neural stem cells or early neurons that have the phenotype of dopaminergic neurons and are capable of structurally and functionally integrating into the host brain following transplantation. Accordingly, these cells are useful in cell replacement/transplantation therapies, including therapies designed to treat Parkinson's disease and other conditions caused by a loss of dopaminergic neurons.

Specifically, fibroblasts are reprogrammed using the four transcription factors Oct4, Sox2, Klf4, and c-Myc. These reprogrammed fibroblasts are then differentiated into functional neurons (“iPS-cell-derived neurons and neuronal precursors”) in vitro. When transplanted into both the normal developing and lesioned brain, these cells differentiate into, and function as midbrain dopaminergic neurons and can restore functional/behavioral deficits caused by dopaminergic denervation.

iPS-cell-derived neurons and neuronal precursors of the present invention may be produced from iPS cells that have been reprogrammed using viral or non-viral methods, and may be produced from human and/or non-human somatic cells. For example, Soldner et al. (Cell, 136: 964-977, 2009; hereby incorporated by reference) produced pluripotent cells using an inducible Cre-Lox (non-viral) system for expressing three (Oct4, Sox2, and KLF4) or four (Oct4, Sox2, KLF4, and c-myc) reprogramming factors in human fibroblasts obtained from patients diagnosed as having Parkinson's Disease. The human pluripotent cells were used to produce embryoid bodies by in vitro culturing in the presence of FGF2, FGF8, and Shh. The cells were terminally-differentiated into a neuronal phenotype expressing dopaminergic cell makers by the withdrawal of growth factors (Soldner et al.).

Mesenchymal Cells

The mesenchymal cells useful for creating iPS cells may be obtained from any suitable source and may be any specific mesenchymal cell type. For example, if the ultimate goal is to generate therapeutic cells for transplantation into a patient, mesenchymal cells from that patient are desirably used to generate the iPS cells. Suitable mesenchymal cell types include fibroblasts (e.g., skin fibroblasts), hematopoietic cells, hepatocytes, smooth muscle cells, and endothelial cells.

Cell Transplantation Therapies

The cells of the present invention are useful for the treatment of any disorder of the central nervous system that is characterized by a loss of dopaminergic neurons and/or would benefit from dopaminergic neuronal cell replacement therapy. Disorders of the nervous system amenable to treatment include, for example, traumatic brain injuries and neurodegenerative diseases including, without limitation, Parkinson's disease.

Cell transplantation therapies typically involve the intraparenchymal (e.g., intracerebral) grafting of the replacement cell populations into the lesioned region of the nervous system, or at a site adjacent to the site of injury. Most commonly, the therapeutic cells are delivered to a specific site by stereotaxic injection. Conventional techniques for grafting are described, for example, in Bjorklund et al. (Neural Grafting in the Mammalian CNS, eds. Elsevier, pp 169-178, 1985), Leksell et al. (Acta Neurochir., 52:1-7, 1980) and Leksell et al. (J. Neurosurg., 66:626-629, 1987). Identification and localization of the injection target regions will generally be done using a non-invasive brain imaging technique (e.g., MRI) prior to implantation (see, for example, Leksell et al., J. Neurol. Neurosurg. Psychiatry, 48:14-18, 1985).

Briefly, administration of cells into selected regions of a patient's brain may be made by drilling a hole and piercing the dura to permit the needle of a microsyringe to be inserted. Alternatively, the cells can be injected into the brain ventricles or intrathecally into a spinal cord region. The cell preparation of the invention permits grafting of the cells to any predetermined site in the brain or spinal cord. It also is possible to effect multiple grafting concurrently, at several sites, using the same cell suspension, as well as mixtures of cells.

Following in vitro cell culture and isolation as described herein, the cells are prepared for implantation. The cells are suspended in a physiologically compatible carrier, such as cell culture medium (e.g., Eagle's minimal essential media), phosphate buffered saline, or artificial cerebrospinal fluid (aCSF). Cell density is generally about 10⁴ to about 10⁷ cells/ml. The volume of cell suspension to be implanted will vary depending on the site of implantation, treatment goal, and cell density in the solution. For example, for treatments in which cells are implanted into the brain parenchyma (e.g., in the treatment of Parkinson's Disease), about 5-60 μl of cell suspension will be administered in each injection. Several injections may be used in each host, particularly if the lesioned brain region is large including, for example, if the cells are transplanted into the caudate nucleus. In contrast, relatively fewer injections are needed if the cells are transplanted into a smaller nucleus (e.g., the substantia nigra). Alternatively, administration via intraventricular injection, for example, will accommodate relatively larger volumes and larger cell numbers (see, for example, Madrazo et al., New Engl. J. Med., 316:831-834, 1987; Penn et al., Neurosurgery, 22:999-1004, 1988).

In some embodiments, the cells are encapsulated within permeable membranes prior to implantation. Encapsulation provides a barrier to the host's immune system and inhibits graft rejection and inflammation. Several methods of cell encapsulation may be employed. In some instances, cells will be individually encapsulated. In other instances, many cells will be encapsulated within the same membrane. Several methods of cell encapsulation are well known in the art, such as described in European Patent Publication No. 301,777, or U.S. Pat. Nos. 4,353,888, 4,744,933, 4,749,620, 4,814,274, 5,084,350, and 5,089,272.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquified using sodium citrate. This creates a single membrane around the cells that is highly permeable to relatively large molecules (MW ˜200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off ˜40-80 kDa).

Methods for Depleting Cell Populations of Undesirable Cell Types

In certain embodiments of the present invention, it is desirable to deplete cell populations of undesirable cell types that contribute to deleterious effects or otherwise possess undesirable properties. Alternatively, cell populations may be depleted of cells which themselves do not possess undesirable properties, but are merely unwanted for the particular end-use of the final cell preparation. Methods for in vitro depletion and/or enrichment of cell populations to select for/against cells expressing certain markers (e.g., cell surface proteins such as SSEA-4) are well known.

Cell Sorting: Fluorescence-activated cell sorting (FACS) is a commonly used cell sorting technique. Cells are sorted based on the ability of fluorescently-labeled antibodies or other markers to bind to the cells of interest. Cells are separated by flow cytometry and sorted into different containers based on their fluorescent characteristics.

Immunomagnetic Cell Separations: Immunomagnetic cell separations involve attaching antibodies directed to cell surface markers (e.g., proteins) to small paramagnetic beads. See, for example, Kruger et al., Transfusion 40: 1489-1493, 2000. When the antibody-coated beads are mixed with the cell sample, the antibodies attach to the cells expressing the marker of interest. The cell sample is then placed in a strong magnetic field, causing the paramagnetic beads (and the bound cells) to pellet to one side. Depending upon the marker of interest, the captured cells may represent either a desirably enriched cell population, with the unbound cells being discarded, or the unbound cells representing the enriched cell population with the unwanted cells removed.

Cellular Panning: For this cellular separation technique, an antibody to the cell type in question is allowed to adhere to a surface, such as the surface of a plastic Petri dish. When the cell mixture is layered on top of the antibody-coated surface, the targeted cells tightly adhere. Non-adherent cells are rinsed off the surface, thereby effecting a cell separation. Cells that express a cell surface protein recognized by the antibody are retained on the plastic surface whereas other cell types are not. This technique is useful for capturing rare cells in a population, but the antibody-bound surface may become saturated and target cells lost in samples having relatively large numbers of target cells.

### EXAMPLE 1

### Neural Cell Production from Reprogrammed Fibroblasts

Nanog-selected iPS cell lines N8, N10 and N14, the Oct4-selected iPS cell lines O9 and 018 (Wernig et al., Nature 448: 318-324, 2007), and the non drug-selected iPS cell line OG-14 (Meissner et al., Nat. Biotechnol. 25: 1177-1181, 2007) were subjected to a multi-stage differentiation protocol, which has been previously developed in ES cells (Lee et al., Nat. Biotechnol. 18: 675-679, 2000) with slight modifications. Briefly, iPS cells were dissociated using trypsin (0.05%) and purified by attachment to tissue culture dishes for one hour. Embryoid bodies (EBs) were allowed 3-4 days to form after plating of iPS cells in bacterial dishes in DMEM media containing 10% defined FBS (Sigma-Aldrich), 2 mM L-glutamine (Invitrogen), 1x NEAA (Invitrogen), 10 mM HEPES (Invitrogen), 1 mM β-mercaptoethanol, 100 U/ml penicillin and 100 μg/ml streptomycin (Invitrogen) (EB media). EBs were allowed one day to attach to tissue culture dishes and neuronal precursor were then selected for by incubation in DMEM/F-12 media containing apotransferrin (50 μg/ml) (Sigma-Aldrich), insulin (5 μg/ml) (Sigma-Aldrich), sodium selenite (30 nM) (Signal-Aldrich), fibronectin (250 ng/ml) (Sigma-Aldrich), 100 U/ml penicillin and 100 μg/ml streptomycin (Invitrogen) (ITSFn media) for 7-10 days. Cells were subsequently dissociated by trypsin (0.05%) and neuronal precursors expanded and patterned for 4 days after plating onto fibronectin-/polyornithine-coated plates at a density of 75,000 cells/cm² in DMEM/F-12 media containing apotransferrin (100 μg/ml), insulin (5 μg/ml), sodium selenite (30 nM), progesterone (20 nM), putrescine (100 nM), penicillin (100 U/ml), streptomycin (100 μg/ml), laminin (1 μg/ml), basic Fibroblast Growth Factor (FGF2) (10 ng/ml) (R&D), Shh (500 ng/ml) (R&D) and FGF8 (100 ng/ml) (R&D) (N3 media). The cells were subsequently differentiated in N3 media containing 200 μM ascorbic acid (AA) for 3-14 days (stage 5). Cells used for immunofluorescent staining were fixed in 4% formaldehyde (Electron Microscopy Sciences, Ft. Washington, Pa.) for 20 min and rinsed with PBS.

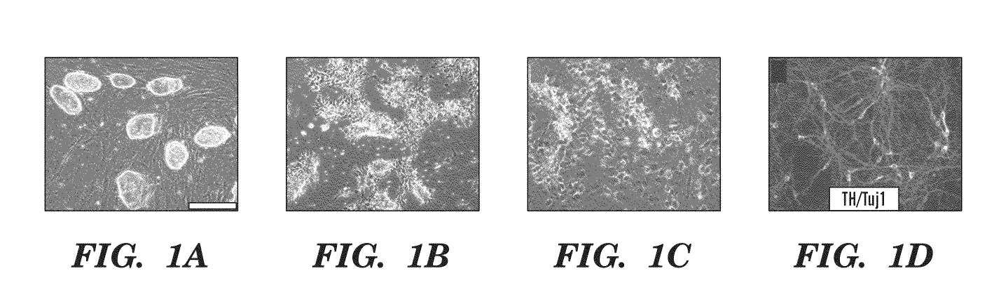

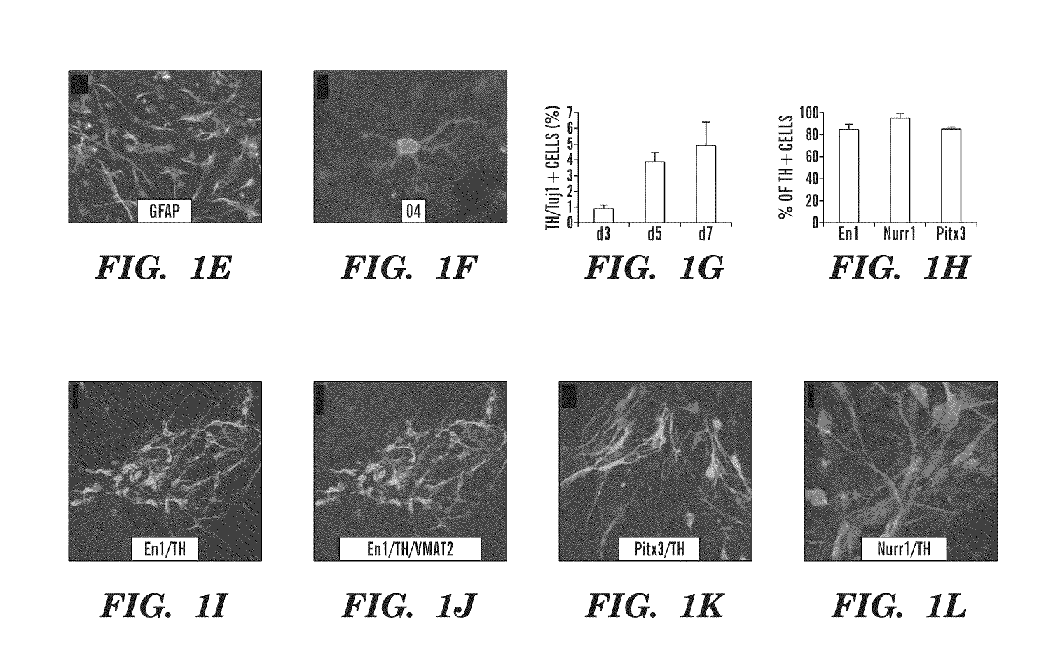

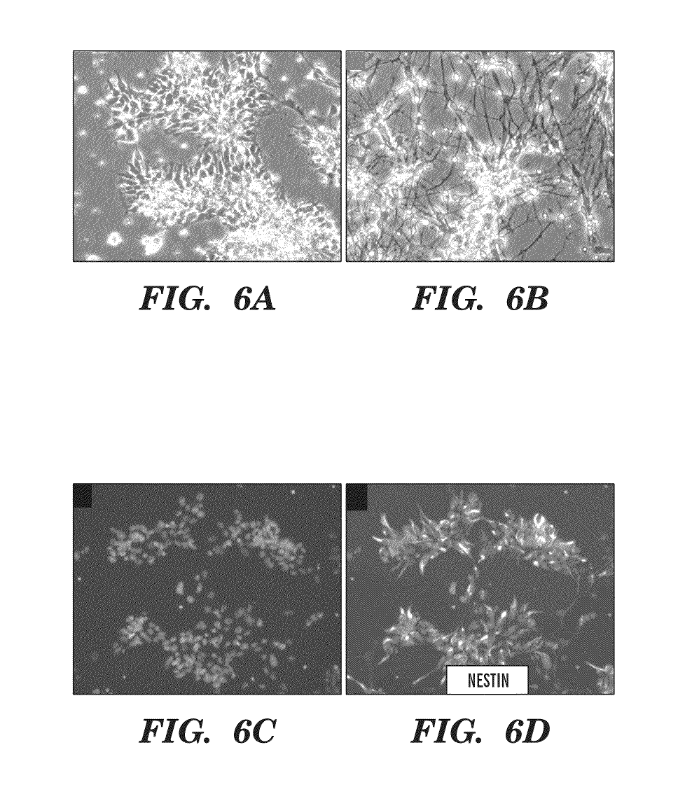

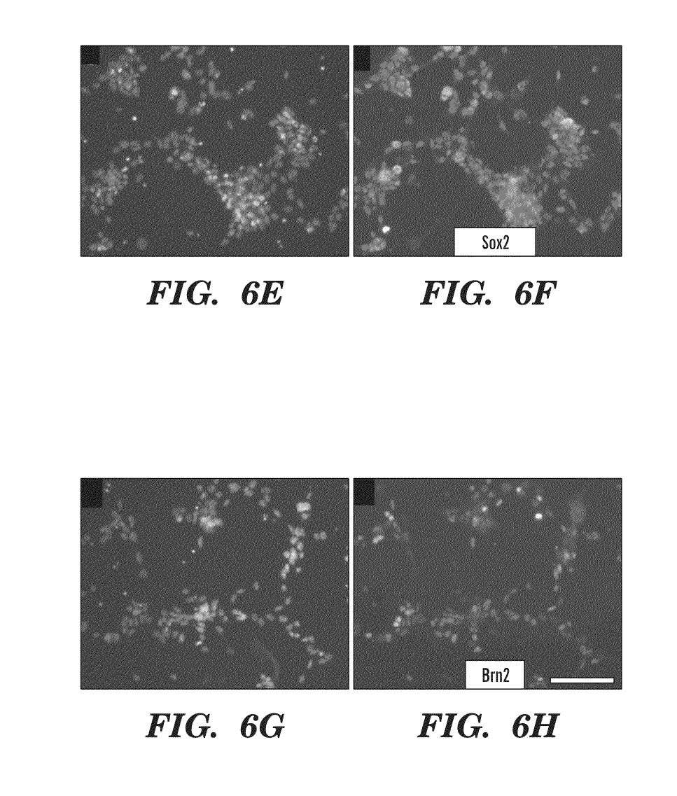

After initial expansion on irradiated MEF feeder cells (FIG. 1A), the iPS cells were passaged onto gelatine-coated dishes to purify from feeder cells and were transferred to non-adherent culture dishes where they readily formed spheroid embryoid bodies. Upon plating and culture in serum-free media the cells formed clusters of neuroepithelial-like cells that were isolated and propagated in FGF2-containing media. These cells displayed a typical neural precursor cell morphology (FIG. 1B) and homogeneously expressed the neural stem cell marker proteins nestin, Sox2, and Brn2 (FIG. 6). Seven days after withdrawal of FGF2, the cells had robustly differentiated into β-III-tubulin-positive neurons, glial fibrillary acidic protein (GFAP)-positive astrocytes and 04-positive oligodendrocytes (FIG. 1C-1F).

These iPS cells were then used to generate neuronal subtypes such as dopamine neurons of midbrain character following protocols developed for mouse ES cells. The FGF2-responsive iPS cell-derived neural precursor cells were then treated with the regional patterning factors sonic hedgehog and FGF8 (Okabe et al., Mech. Dev. 59: 89-102, 1996; Kim et al., Nature 418: 50-56, 2002; Lee et al., Nat. Biotechnol. 18: 675-679, 2000). After withdrawal of the growth and patterning factors most cells differentiated into β-III-tubulin-positive cells with neuronal morphology, a fraction of which could also be labeled with antibodies against tyrosine hydroxylase (TH) (FIG. 1D). Quantification of three independent experiments revealed that the number of TH-positive neurons increased over time in culture (FIG. 1G). These cells also expressed the vesicular monoamine transporter 2 (VMAT2) that is responsible for catecholamine transport into synaptic vesicles (FIG. 1J). The cells were further characterized for a dopaminergic phenotype by double labeling for tyrosine hydroxylase (TH) and En1, Pitx3, or Nurr1; all markers typically expressed in dopamine neurons of the midbrain. As shown in FIGS. 11, 1K, and 1L, the vast majority of TH-positive cells stained for these three midbrain markers suggesting their proper regional specification in vitro.

### EXAMPLE 2

### iPS-Derived Neural Precursor Cells Migrate and Differentiate into Neurons and Glia Following Transplantation

Neural precursor cells were derived from iPS cells that had been infected with a GFP-expressing lentivirus (Lois et al., Science 295: 868-872, 2002). About 100,000-300,000 FGF2-responsive neural precursor cells derived from the GFP-positive iPS cell subclones N8.2, N14.2, and O9.2 were transplanted in utero into the lateral brain ventricles of E13.5-E14.5 mouse embryos. The surgical procedures have been described previously (Brustle et al., Neuron 15: 1275-1285, 1995; Brustle et al., Proc. Natl. Acad. Sci. USA 94: 14809-14814, 1997; Bjorklund et al., Proc. Natl. Acad. Sci. USA 99: 2344-2349, 2002). Transplanted mice were spontaneously delivered and analyzed one to nine weeks after surgery. The brains were serially sectioned and cells incorporated into the brain parenchyma (located at least 50 μm from clusters or the ventricular wall) were counted, morphologically assessed, and functionally analyzed using electrophysiological techniques.

Morphological Assessment

For immunofluorescent staining, cells on coverslips and tissue sections were rinsed with PBS and incubated with blocking buffer (PBS, 10% normal donkey serum; NDS or normal goat serum; NGS, 0.1% Triton-X100) for 1 h. Coverslips/sections were then incubated overnight at 4° C. with primary antibodies diluted in PBS, 10% NDS/NGS, 0.1% Triton-X100). The following primary antibodies were used: rabbit anti-GFP (1:1,000; Molecular Probes, Invitrogen), sheep anti-TH P601010 (1:1,000) and rabbit anti-vesicular monoamine transporter 2 (VMAT2; 1:1,000; Pel-Freez Biologicals, Rogers, Ariz.), sheep antiaromatic L-amino acid decarboxylase (AADC; 1:200), mouse anti-GAD67 MAB5406 (1:100), rabbit anti-EAAC1 (1:100), mouse anti-04 (1:50), mouse anti-NeuN (1:50) and mouse anti-nestin (clone rat-401; 1:100; Chemicon, Millipore), rabbit anti-paired-like homeodomain transcription factor 3 (Pitx3; 1:250, Zymed), mouse anti-Synaptophysin (1:40), rabbit anti-GFAP (1:500; Dako, Carpinteria. Calif.), rabbit anti-Nurr1 (E-20; 1:300), goat anti-Brn2 (1:50; Santa Cruz Biotechnology, Santa Cruz, Calif.), mouse antiengrail-1 (1:40) and rabbit anti-Ki67 (1:2,000; Novocastra), rabbit anti-Nanog (1:100; Bethyl), and mouse anti-Sox2 (1:100, R&D Systems). The coverslips/tissue sections were subsequently incubated in fluorescent-labeled secondary antibodies (Jackson Immunoresearch Laboratory) in PBS and 10% NDS/NGS for 1 h at room temperature. After rinsing for 3×10 min in PBS, Hoechst 33342 (4 mg/ml) was used for counterstaining and coverslips/tissues sections were mounted onto slides in Gel/Mount (Biomeda Corp., Foster City, Calif.). Control experiments were performed by omission of primary antibodies and using different combinations of secondary antibodies. Confocal analysis was performed using a Zeiss LSM510/Meta Station (Thornwood, N.Y.). For identification of signal colocalization within a cell, optical thickness was kept to a minimum, and orthogonal reconstructions were obtained. Stereology was performed using Stereo Investigator image-capture equipment and software (MicroBrightField, Willinston, Vt.) and a Zeiss Axioplan I fluorescent microscope. Graft volumes were calculated using the Cavalieri estimator probe. A minimum of three coverslips was counted for each immunostaining

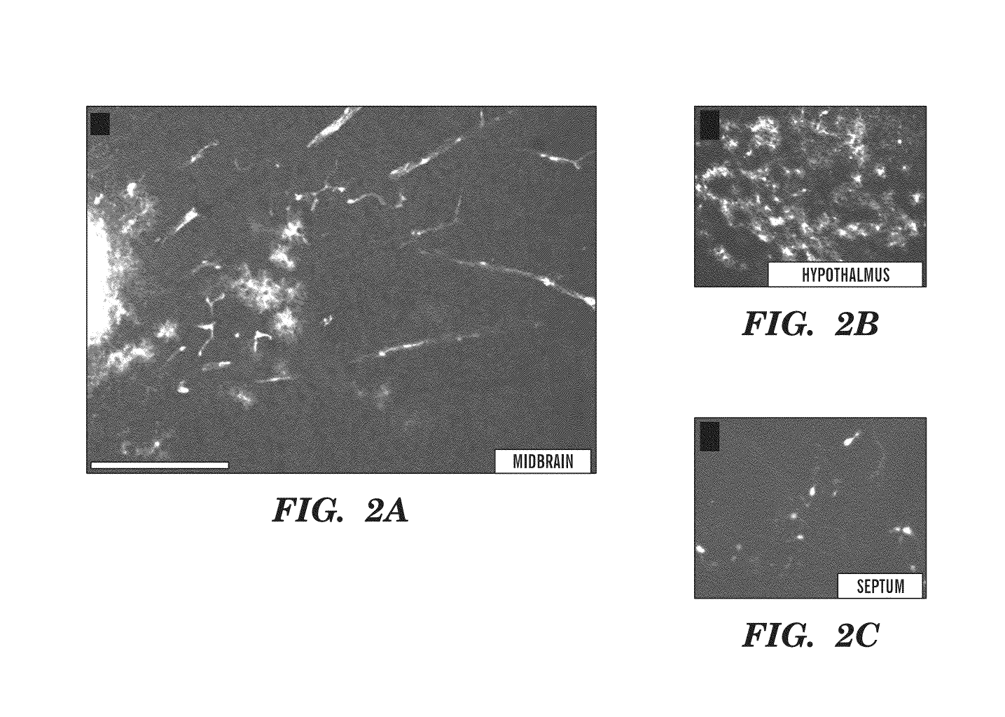

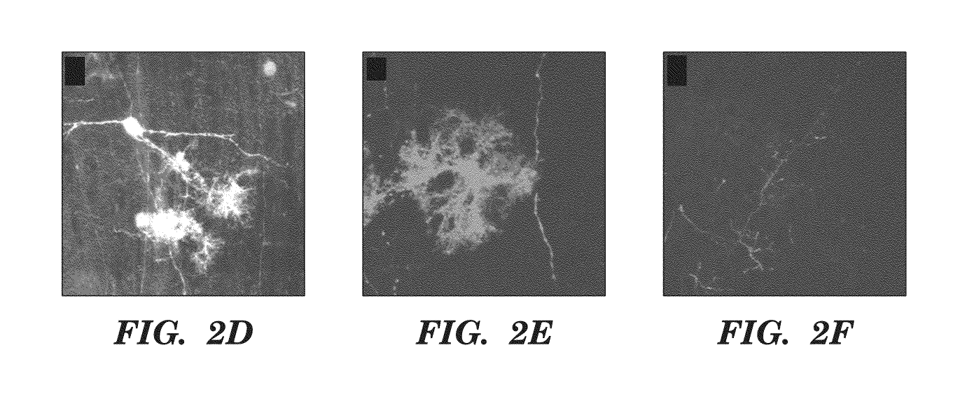

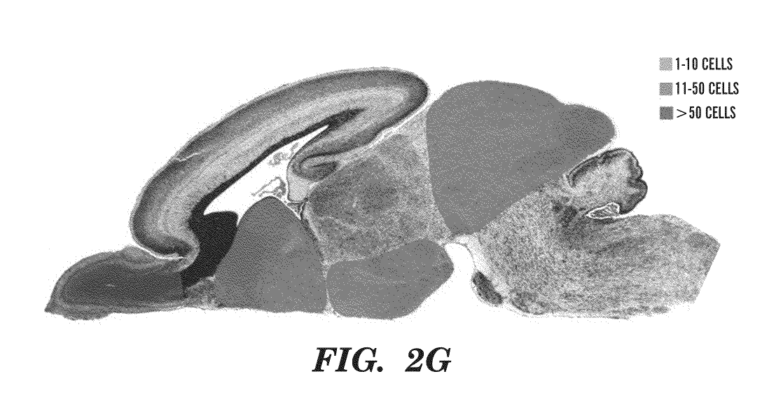

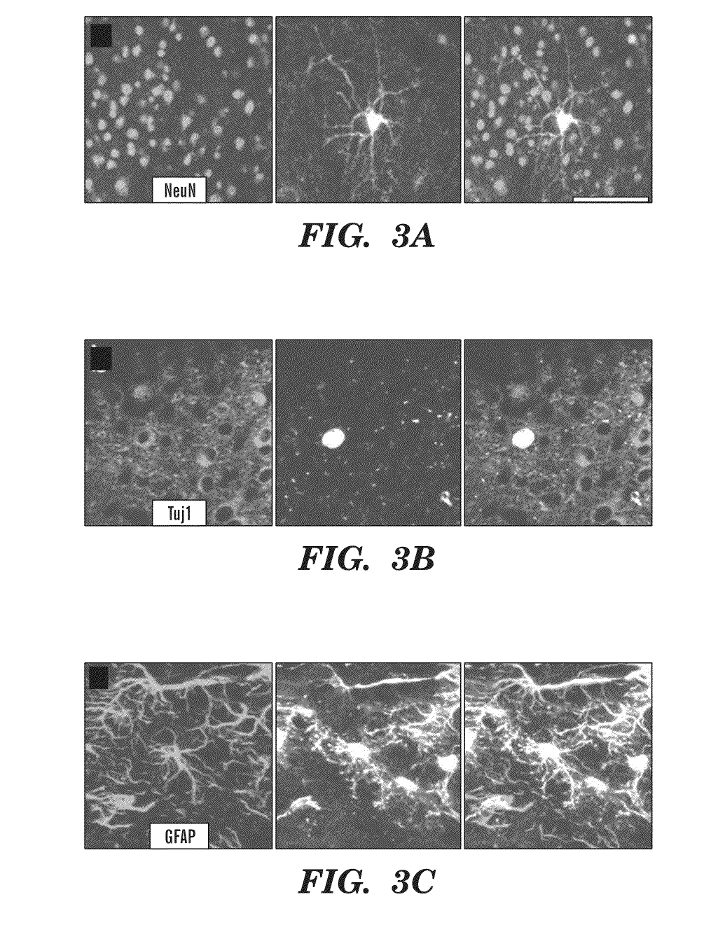

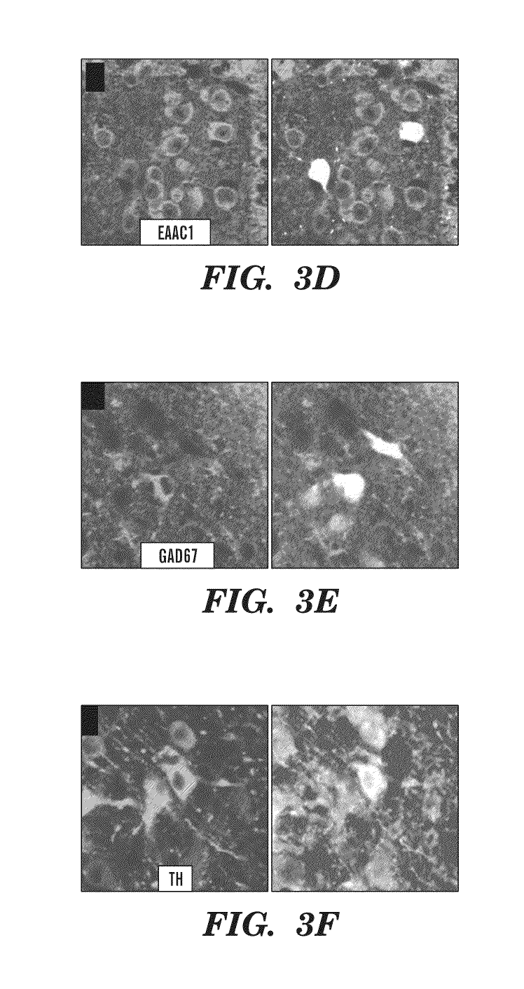

As shown in FIG. 2A, transplanted cells formed intraventricular clusters and some had migrated extensively into the surrounding brain tissue. GFP-positive cells were found in many different brain regions. The highest densities of transplanted cells were found in septum, striatum, hypothalamus and midbrain. Smaller numbers were detected in olfactory bulb, cortex and thalamus and no cells were found in cerebellum and brain stem (FIG. 2A-2C, 2G, and Table 1). Incorporated cells displayed various complex neuronal and glial morphologies (FIG. 2C-F) expressing the neuronal marker proteins NeuN and β-III-tubulin or the glial marker GFAP (FIG. 3A-3C). The engrafted neurons gave rise to various neuronal subtypes including glutamate transporter EAAC1-positive glutamatergic neurons, Glutamic acid decarboxylase 67 (GAD67)-positive GABAergic neurons and TH-positive catecholaminergic neurons (FIG. 3D-3F).

TABLE 1
Incorporation of iPS cell-derived neurons and glia
after in utero transplanation

| Animal | Age | OB | CTX | SPT | TH | HT | MB | CB/BS |
|---|---|---|---|---|---|---|---|---|
| 329.1 | P0 | ++ | + | −− | + | −− | ++ | −− |
| 329.2 | P0 | −− | + | −− | −− | −− | −− | −− |
| 1855.1 | P0 | −− | − | −− | + | + | −− | −− |
| 1856.3 | P0 | −− | −− | + | −− | + | −− | −− |
| 1870.1 | P27 | −− | ++ | + | −− | ++ | −− | −− |
| 1865.1 | P29 | −− | ++ | +++ | −− | +++ | +++ | −− |
| 1865.3 | P29 | −− | ++ | ++ | + | +++ | −− | −− |
| 1857.2 | P56 | −− | + | ++ | ++ | + | −− | −− |
| 1857.3 | P56 | −− | + | ++ | −− | −− | −− | −− |
| 1857.4 | P56 | −− | ND | ND | ND | ND | +++ | −− |

Indicated are the maximum number of cells on a 50-μm section from at least three sections per brain region.
−− = no cells;
+ = 1-10 cells;
++ = 11-50 cells;
+++ = >50 cells.
OB = olfactory bulb;
CTX = cortex;
SPT = septum;
TH = thalamus;
HT = hypothalamus;
MB = midbrain;
CB = cerebellum;
BS = brain stem;
ND = not done.

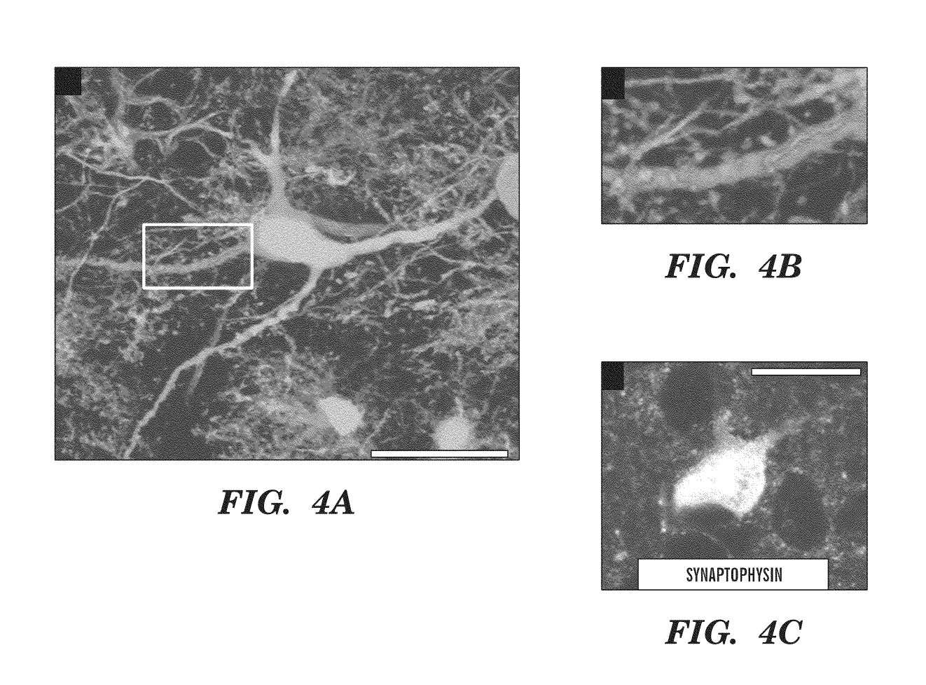

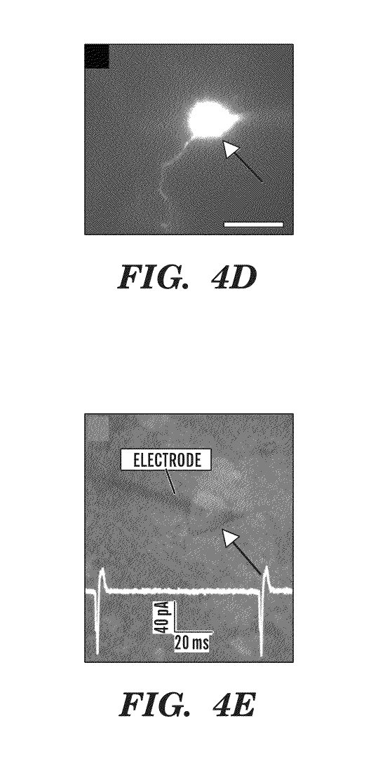

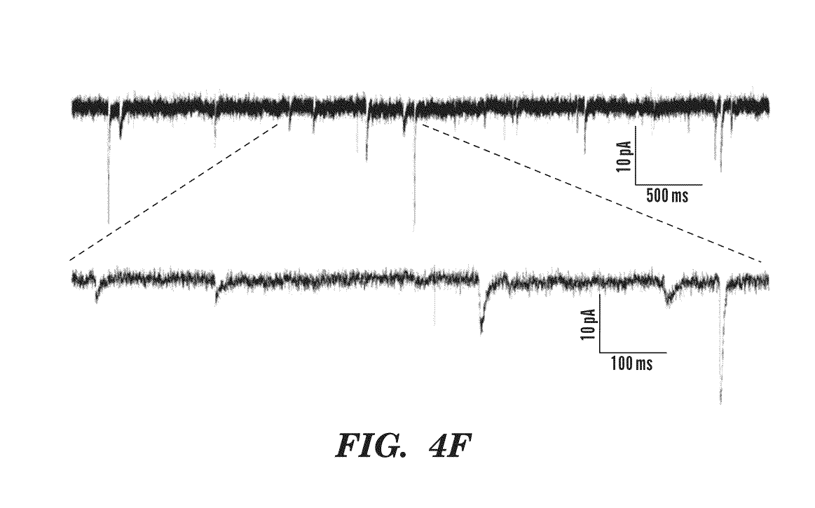

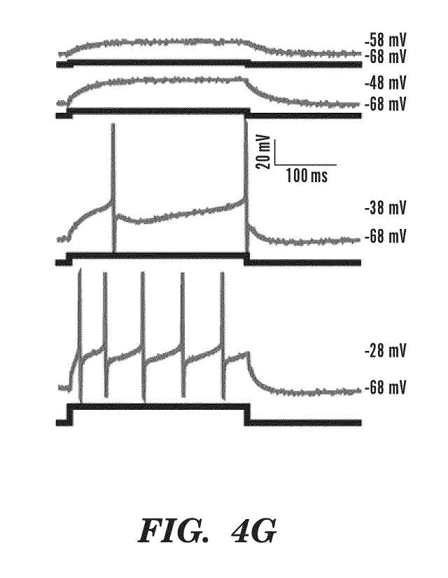

Neuronal maturity and synaptic integration of transplanted iPS cell-derived neurons was determined by morphological criteria. Immunofluorescent labeling for GFP provided a crisp outline of the incorporated cells, clearly delineating their shapes and fine neuronal processes (FIGS. 2E-2F and FIGS. 4A-4B). Confocal analysis demonstrated the presence of small synaptic spines on the surface of dendritic processes and numerous synaptophysin-positive, GFP-negative patches were found in close apposition to the somatic and dendritic membranes of transplanted cells, suggesting that host-derived presynaptic terminals contacted iPS cell-derived neurons (FIG. 4C).

Electrophysiological Assessment

Electrophysiological recordings from brain slices prepared from transplanted animals were used to examine functional neuronal properties in the engrafted cells. P20 and P22 mice with embryonic stem cell injections were anesthetized with isoflurane and the brains removed. The midbrain was dissected and placed in ice-cold artifact CerebroSpinal Fluid (ACSF) containing the following (in mM): 124 NaCl, 3 MgCl₂, 4 KCl, 3 CaCl₂, 1.25 NaHPO₄, 26 NaHCO₃, and 16 D-glucose saturated with 95% O2/5% CO₂ to a final pH of 7.35. Parasagittal slices (350 μm thick) were cut on a vibratome and incubated in 32-34° C. ACSF for at least 1 h before recordings. Slices were transferred to a recording chamber on the stage of an upright microscope (Nikon E600FN, Tokyo, Japan) with a 60× water-immersion objective and perfused with room temperature ACSF. GFP-positive neuron-like cells were identified using a fluorescence camera (CoolSNAP EZ, Photometrics, Germany), and were subsequently visualized using infrared differential interference contrast optics (IR DIC). Pipette electrodes (3-5 MΩ resistance) were pulled from borosilicate glass capillaries. The pipette solution contained the following (in mM): 105 K-gluconate, 30 KCl, 10 phosphocreatine, 10 HEPES, 4 ATP-Mg, 0.3 GTP, 0.2 EGTA, pH adjusted to 7.3 with KOH and osmolarity adjusted to 298 mOsmol with sucrose. Series resistances were always <40 MΩ, electrical signals were amplified with an Axonpatch 200B amplifier, digitized with a Digidata 1322A interface (Molecular Devises, Union City, Calif.) and filtered at 2 kHz, sampled at 10 kHz.

GFP-positive cells with long dendrite-like processes were identified as neurons by focusing through the depth of the tissue (FIG. 4D). All cells recorded from two animals were in the central region of the inferior colliculus. To properly place the electrode, the microscope was switched to infrared differential interfercence contrast (IR DIC) in the plane of the cell body (FIG. 4E). Cell-attached voltage-clamp recordings were made in three GFP-positive cells. All three cells showed spontaneous action potential currents (FIG. 4E).

Synaptic inputs were examined in an additional six cells held in voltage clamp at −70 mV. All cells showed spontaneous postsynaptic currents ranging in amplitude and kinetics and therefore indicative of inputs from different ionotropic transmitter receptor types. At −70mV, with our recording solutions, inward currents will reflect both inhibitory and excitatory synaptic activity (FIG. 4F). To test the active membrane characteristics of the labeled cells recordings were switched to current clamp mode. Resting membrane potentials of these cells ranged from −53 to −63 mV (−60±2.4 mV).

For current injection experiments the resting membrane potential was shifted to the more polarized potential of −68 mV. Starting from this potential, depolarizing current injections induced action potentials ranging in amplitude from 70 to 82 mV (78.8±2.9 mV). Thresholds for action potential initiation were in the range of −40 mV (FIG. 4G).

### EXAMPLE 3

### Transplantation of iPS Cell-Derived Midbrain Dopaminergic Neurons Results in a Functional Recovery from a 6-OHDA Lesion

One of the prime candidate diseases for cell replacement therapy is Parkinson's disease due to the localized degeneration of a specific cell type; the A9 dopaminergic neurons. As shown above, transplanted neurons from in vitro-generated from iPS cells were functionally integrated into the host brain. The following experiment demonstrates that iPS cell-derived neurons are capable of restoring the functional deficits caused by the selective loss of midbrain dopaminergic neurons.

Adult female Sprague-Dawley rats (200-250 g; Taconic) were unilaterally lesioned by 6-hydroxydopamine (6-OHDA) injection (8 μg, 2 μg/μL/min) into the medial forebrain bundle (AP-4.3, Lat-1.2, DV-8.3) under sodium pentobarbital anesthesia. Rotational behavior in response to amphetamine (4 mg kg i.p.) was evaluated before and 4 weeks after 6-OHDA lesion. Animals were placed (randomized) into automated rotometer bowls, and left and right full-body turns were monitored by a computerized activity monitor system. Animals showing >600 turns ipsilateral to the lesioned side in 90 min after a single dose of amphetamine (average 10.2±0.7 turns/min) were selected for transplantation. Two groups of either sham-operated rats (n=10) or of lesioned-only rats (n=10) matched for the severity of baseline amphetamine rotation served as controls (n=10).

Reprogrammed fibroblasts (iPS cell clone O9) were differentiated into dopamine neurons as described above and animals lesioned with 6-OHDA either received a sham operation or a striatal graft of 1-3×10⁵ differentiated cells 5 days after the cells were withdrawn from the growth and patterning factors (stage 5, day 5).

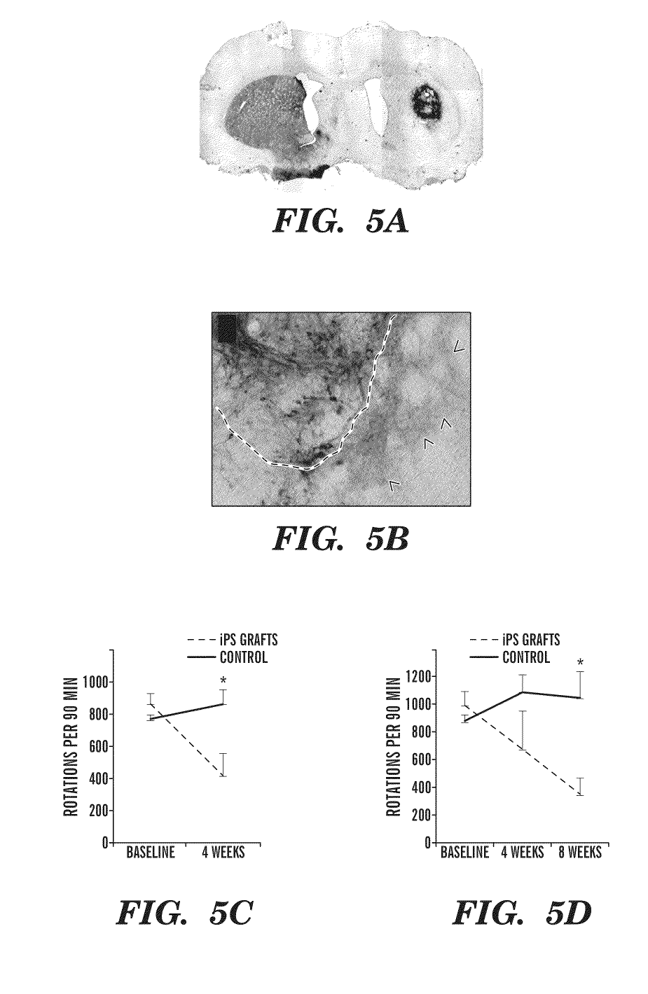

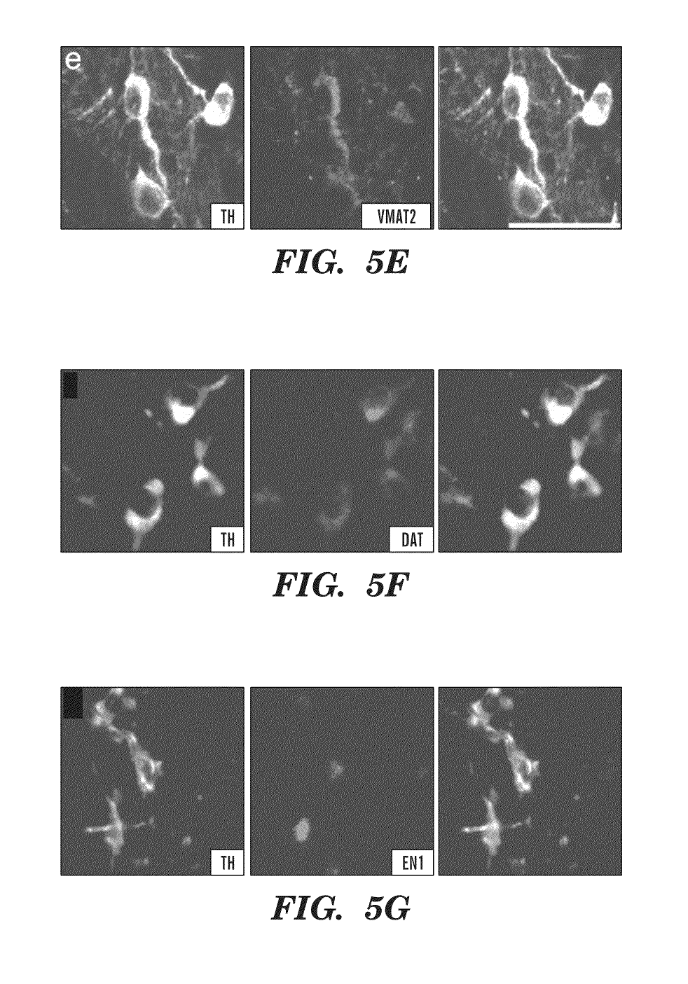

Four weeks after surgery animals were used for morphological analysis with TH immunostaining. Sham-grafted animals showed no TH-positive elements in the ipsilateral substantia nigra or the dorsal striatum. In contrast, in the striatum of rats grafted with differentiated iPS cells a large number of TH-positive cells were found (FIG. 5A). These cells showed complex morphologies (FIG. 5B) and were also positive for En1, VMAT2, and dopamine transporter (DAT) (FIG. 5E-5G). The somata of TH-positive cells remained in close vicinity of the graft but TH-immunoreactive fibers were found to extend into the parenchyma of the host striatum (FIG. 5B, dashed line delineates the border-zone of the graft).

The behavior of sham-operated rats and rats grafted with iPS cell-derived dopaminergic neurons was examined. Amphetamine stimulation to animals lesioned unilaterally with 6-OHDA induces a movement bias ipsilateral to the injection site. Whereas the control group showed a stable rotational bias over time, 4 out of 5 transplanted animals showed a marked recovery of the rotation behavior 4 weeks after transplantation (FIG. 5B). All four responding animals contained large numbers of TH-positive neurons in contrast to the one non-responding animal. Cell counts in serial sections from one representative responding animal revealed that the graft contained an estimated total number of about 29,000 TH-positive neurons whereas only about 1,500 TH-positive cells were estimated to have been present in the non-responding animal. In the latter animal, despite a relatively high number of DA neurons in the large graft, they were typically located in the center of the graft, and so very few DA fibers extended to the host striatum. Consequently, only marginal innervation (≦10%) of the dopamine-depleted striatum was achieved, which might be the reason for lack of functional recovery at this time point.

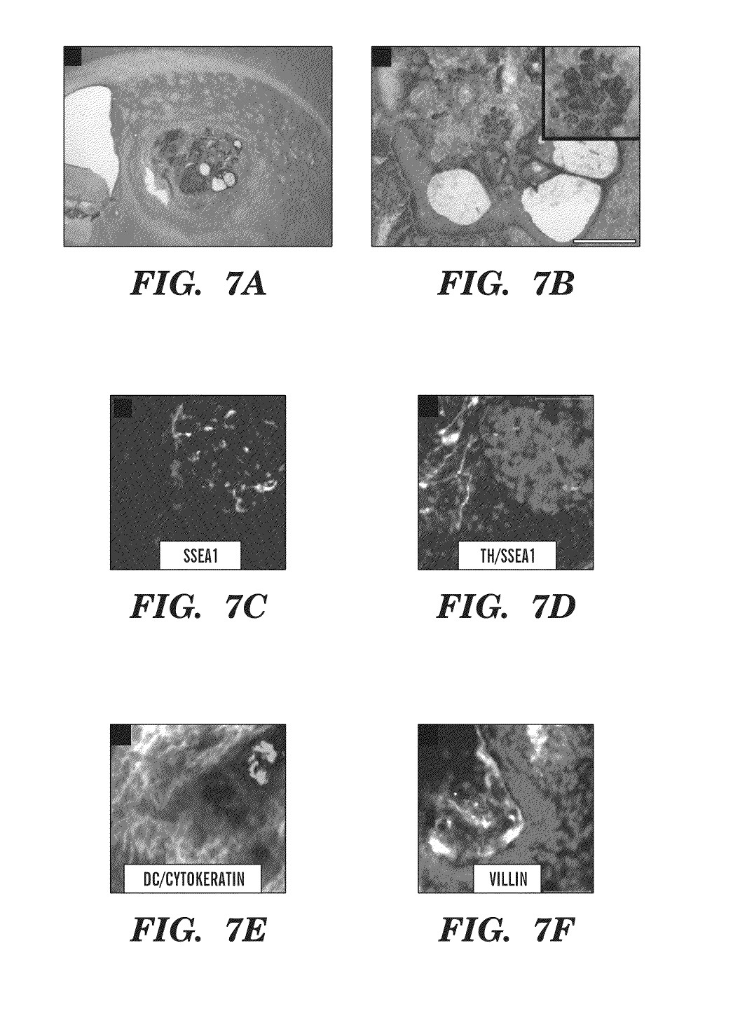

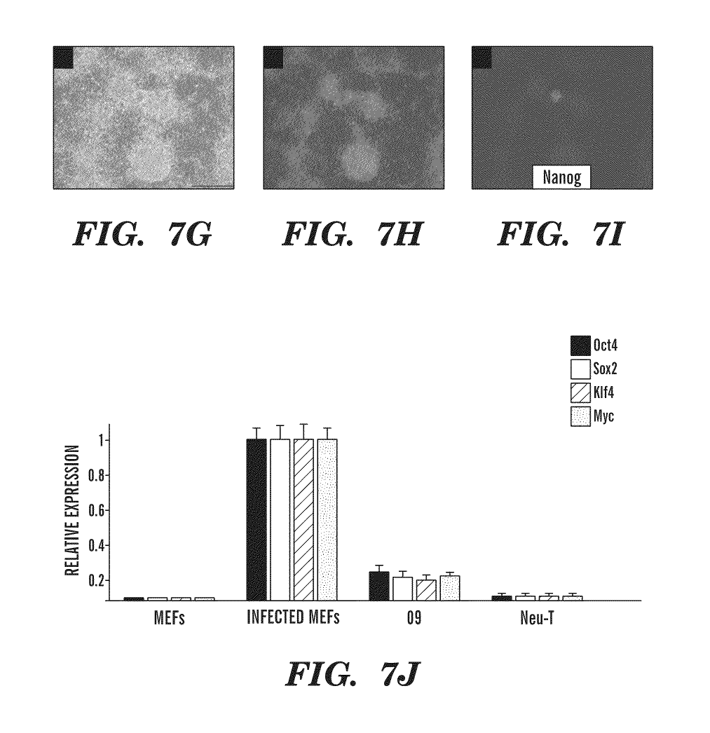

Immunohistochemical examination revealed graft areas containing Ki67-positive cells in all five animals that showed functional recovery and in 2/5 animals from another set of transplantations indicating the continuous proliferation of transplanted cells. Upon close morphological examination, we identified histological structures of non-neural tissue suggesting the presence of teratoma formations (FIGS. 7A-7F). The contamination of undifferentiated ES cells and subsequent teratoma formation after transplantation still appears to be a major complication of ES cell-based therapies in animal transplantation models. This seems the most likely reason for teratoma formation also in our experiments as the viral transcripts were not reactivated in those tumors (FIG. 7J).

Reanalysis of the iPS cell cultures at the stages used for transplantation (-3 weeks of differentiation) did identify rare and small clusters of undifferentiated Nanog-positive cells although the vast majority of these cultures contained postmitotic neurons (FIGS. 7G-7I). These findings suggest that elimination of undifferentiated cells from the cultures should reduce the risk of teratoma formation after transplantation.

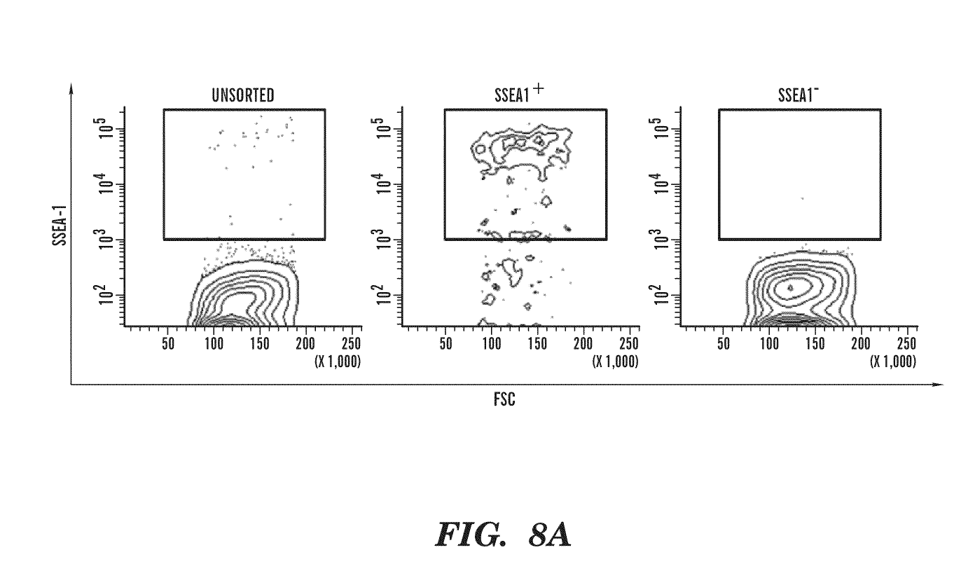

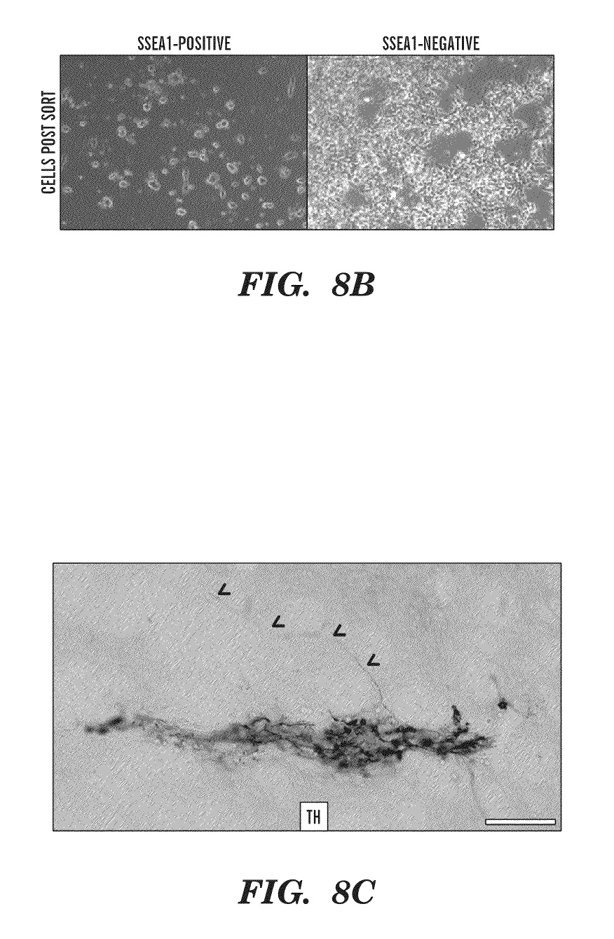

Accordingly, fluorescence-activated cell sorting (FACS) was used to deplete the cell suspension from SSEA1-positive cell fraction prior to transplantation (FIG. 8A). Cultures established from sorted cells showed a reduced presence of undifferentiated cell types, and a network of differentiated neurons as soon as one day after plating (FIG. 8B). Four animals grafted with iPS cell-derived neuronal cell preparations depleted of SSEA1-positive cells recovered at degrees similar to animals receiving non-purified cell suspensions (FIG. 5D). Histologically, the grafts were consistently smaller and no tumor formation was observed up to 8 weeks after transplantation (FIG. 8C).

Unless otherwise defined, all technical and scientific terms used herein have the same meaning as commonly understood by one of ordinary skill in the art to which this invention belongs.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for producing neural stem cells comprising:
(i) providing a pluripotent stem cells that were derived from mesenchymal cells; and
(ii) obtaining neural stem cells by culturing said induced pluripotent stem cells in the presence of at least one neural selection factor.

2. The method of claim 1, wherein the pluripotent stem cells were produced by overexpressing in at least one transcription factor selected from the group consisting of Oct4, Sox2, c-Myc and Klf4

3. The method of claim 1, wherein each of Oct4, Sox2, c-Myc and Klf4 is overexpressed in said mesenchymal cells.

4. The method of claim 1, wherein said at least one of said neural selection factors is selected from the group consisting of SHH, FGF-2, and FGF-8.

5. The method of claim 1, wherein said mesenchymal cells are human mesenchymal cells.

6. The method of claim 5, wherein said mesenchymal cells are fibroblasts.

7. The method of claim 6, wherein said fibroblasts are skin fibroblasts.

8. The method of claim 1, wherein said neural stem cells express nestin.

9. A population of neural stem cells produced by the method of claim 1.

10. The population of neural stem cell of claim 9, wherein at least 50% of the cells of said population expresses nestin.

11. The population of neural stem cell of claim 10, wherein said nestin-expressing cells further express at least one protein selected from the group consisting of tyrosine hydroxylase, DAT, VMAT, En-1, Pitx3, and Nurr-1.

12. The population of neural stem cells of claim 9, wherein said population has been depleted of cells expressing SSEA-4.

13. A population of neural stem cells derived from induced pluripotent stem cells, wherein said population has been depleted of at least 50% of the cells expressing SSEA-4.

14. The population of neural stem cells of claim 13, wherein said population contains no more than 5% SSEA-4-positive cells.

15. The population of neural stem cells of claim 14, wherein said population contains no more than 1% SSEA-4-positive cells.

16. A therapeutic composition comprising a cell population of claim 9.

17. The therapeutic composition of claim 16, wherein said population of cells is suspended in a physiologically compatible solution.

18. The therapeutic composition of claim 17, wherein said solution is artificial cerebrospinal fluid.

19. The therapeutic composition of claim 16, wherein said population of cells is encapsulated.

20. The therapeutic composition of claim 16, wherein said population of cells is contained within an inert biomatrix.

21. A method for treating a neurodegenerative disease in a patient, comprising administering to the brain of said patient a therapeutic composition of claim 16.

22. The method of claim 21, wherein said neurodegenerative disease is Parkinson's disease.

23. The method of claim 22, wherein said therapeutic composition is injected into the striatum of said patient.

24. The method of claim 22, wherein said therapeutic composition is injected into the midbrain of said patient.

25. The method of claim 21, wherein said mesenchymal cells are obtained from the patient.
